(19) United States
(12) Patent Application Publication
(10) Pub. No.: US 2008/0108856 A1
(43) Pub. Date: May 8, 2008

(54) Process For The Manufacture Of 1,2-Dichloroethane

(76) Inventors: Michel Strebelle, Brussels (BE); Dominique Balthasart, Brussels (BE)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/722,598

(22) PCT Filed: Dec. 21, 2005
(86) PCT No.: PCT/EP05/57046

§ 371 Date: Oct. 15, 2007

(30) Foreign Application Priority Data
Dec. 23, 2004 (FR) 04.13873
Apr. 1, 2005 (FR) 05.03252
Apr. 1, 2005 (FR) 05.03253

(51) Int. Cl. C07C 17/08

(52) U.S. Cl. 570/243

(57) ABSTRACT

Process for the manufacture of 1,2-dichloroethane starting with a hydrocarbon source according to which: a) the hydrocarbon source is subjected to a first cracking step, namely a pyrolysis step carried out in a cracking oven, thus producing a mixture of cracking products; b) the said mixture of cracking products is subjected to a succession of treatment steps which make it possible to obtain a mixture of products containing ethylene and other constituents, among which an aqueous quenching step, an alkaline washing step aimed at removing at least most of the carbon dioxide generating an alkaline solution and an oxidation step aimed at removing the hydrogen sulphide contained in the mixture of cracking products; c) the mixture of products containing ethylene derived from step b) is separated into at least one fraction containing ethylene and into a heavy fraction; d) the fraction(s) containing ethylene is (are) conveyed to a chlorination reactor and/or an oxychlorination reactor, in which reactors most of the ethylene present is converted to 1,2-dichloroethane; e) the 1,2-dichloroethane obtained is separated from the streams of products derived from the chlorination and oxychlorination reactors.

The present invention relates to a process for the manufacture of 1,2-dichloroethane (DCE), a process for the manufacture of vinyl chloride (VC) and a process for the manufacture of polyvinyl chloride (PVC).

To date, ethylene which is more than 99.8% pure is normally used for the manufacture of DCE. This ethylene of very high purity is obtained via the cracking of various petroleum products, followed by numerous complex and expensive separation steps in order to isolate the ethylene from the other products of cracking and to obtain a product of very high purity.

Given the high cost linked to the production of ethylene of such high purity, various processes for the manufacture of DCE using ethylene having a purity of less than 99.8% have been developed. These processes have the advantage of reducing the costs by simplifying the course of separating the products resulting from the cracking and by thus abandoning complex separations which are of no benefit for the manufacture of DCE.

The products leaving the first cracking step, namely the pyrolysis step carried out in a cracking oven, are conventionally subjected to a succession of treatment steps such as an aqueous quenching in order to condense the water contained in the products and an alkaline washing aimed at removing the hydrogen sulphide (H₂S) and the carbon dioxide (CO₂) contained in the products. The first is a toxic contaminant while the second poses a problem of formation of solids in the cold areas under high pressure which are used for the downstream separation of the cracking products.

The presence of sulphur may result from a contamination of the hydrocarbon source to be cracked such as the use of sulphur additives during the supply of the cracking oven.

It is desired to remove the H₂S which, apart from its toxicity, could contaminate the catalysts used in the steps of chlorination or oxychlorination of ethylene to DCE if it were carried with the ethylene. The activities of these catalysts, which are generally respectively based on iron and copper chlorides, would be affected by formation of the corresponding sulphides or sulphates.

The conventional method used in the crackings consists in an alkaline washing with a strong base such as sodium hydroxide (NaOH) which is necessary to fix the weak acids such as H₂S and CO₂.

Moreover, the production of DCE consumes basic solutions in order to neutralize the acidic effluents. A well-known case is the washing of the crude gases leaving an oxychlorination. It is desired to fix the unconverted hydrogen chloride (HCl) in order to avoid problems of corrosion downstream of the equipment. The use of an alkali loop which supplies any device for gas-liquid contact (spray column, ejector followed by a section for gas-liquid separation) is interesting.

In the context of a coupling of a cracking and a VCM unit, it is desired to upgrade the solution resulting from the alkaline washing of the hydrocarbons in order to neutralize the HCl not converted during the oxychlorination. To do this, it is therefore necessary to destroy the H₂S contained in the cracking products or in this alkaline solution.

The subject of the present invention is therefore a process for the manufacture of DCE starting with a hydrocarbon source according to which:
a) the hydrocarbon source is subjected to a first cracking step, namely a pyrolysis step carried out in a cracking oven, thus producing a mixture of cracking products;
b) the said mixture of cracking products is subjected to a succession of treatment steps which make it possible to obtain a mixture of products containing ethylene and other constituents, among which an aqueous quenching step, an alkaline washing step aimed at removing at least most of the carbon dioxide generating an alkaline solution and an oxidation step aimed at removing the hydrogen sulphide contained in the mixture of cracking products;
c) the mixture of products containing ethylene derived from step b) is separated into at least one fraction containing ethylene and into a heavy fraction;
d) the fraction(s) containing the ethylene is (are) conveyed to a chlorination reactor and/or an oxychlorination reactor, in which reactors most of the ethylene present is converted to 1,2-dichloroethane;
e) the 1,2-dichloroethane obtained is separated from the streams of products derived from the chlorination and oxychlorination reactors.

The expression hydrogen sulphide is understood to mean the hydrogen sulphide itself, but also the other sulphides which may be present in the medium in traces, such as for example CS₂ and COS.

The hydrocarbon source considered may be any known hydrocarbon source. Preferably, the hydrocarbon source subjected to cracking (step a)) is chosen from the group consisting of naphtha, gas oil, natural gas liquid, ethane, propane, butane, isobutane and mixtures thereof. In a particularly preferred manner, the hydrocarbon source is chosen from the group consisting of ethane, propane and propane/butane mixtures. Good results were obtained with a hydrocarbon source chosen from the group consisting of propane and propane/butane mixtures. The propane/butane mixtures may exist as such or may consist of mixtures of propane and butane.

The expression ethane, propane, butane and propane/butane mixtures is understood to mean, for the purposes of the present invention, products that are commercially available, namely that consist mainly of the pure product (ethane, propane, butane or propane/butane as a mixture) and secondarily of other saturated or unsaturated hydrocarbons, which are lighter or heavier than the pure product itself.

The expression first cracking step, namely a pyrolysis step carried out in a cracking oven (step a)), is understood to mean a conversion, under the action of heat, of the hydrocarbon source in the presence or absence of third compounds such as water, oxygen, a sulphur derivative and/or a catalyst so as to give rise to the formation of a mixture of cracking products.

This mixture of cracking products advantageously comprises hydrogen, carbon monoxide, carbon dioxide, nitrogen, oxygen, hydrogen sulphide, organic compounds comprising at least one carbon atom and water.

This first cracking step is advantageously followed by step b) consisting of a succession of treatment steps among which are the steps for thermal recovery of the heat of the cracked gases, optionally organic quenching (optionally including recovery of heat through a succession of exchangers with intermediate fluids), aqueous quenching, compression and drying of the gases, alkaline washing aimed at removing at least the majority of the carbon dioxide generating an alkaline solution, optionally hydrogenating the undesirable derivatives such as, for example, acetylene, optionally removing part of the hydrogen and/or the methane and oxidation aimed at removing H₂S. The aqueous quenching step advantageously precedes the alkaline washing step.

According to the first variant of the process according to the invention, the oxidation step aimed at removing the H₂S advantageously consists in the destruction of H₂S via the introduction of an oxidizing agent at the aqueous quenching step. The aqueous quenching and alkaline washing steps may then be separate steps or may be combined. They are preferably two separate steps. In a particularly preferred manner, the aqueous quenching step precedes the alkaline washing step.

Any oxidizing agent may be used. There may be mentioned in particular hydrogen peroxide, sodium hypochlorite and chlorine oxides. Hydrogen peroxide and sodium hypochlorite are however preferred with a most particular preference for hydrogen peroxide.

According to this first variant, when sodium hypochlorite is used as oxidizing agent, it is advantageously used in a sodium hypochlorite:hydrogen sulphide weight ratio ranging from 5:1 to 15:1. Preferably, it is used in a sodium hypochlorite:hydrogen sulphide weight ratio ranging from 8:1 to 9:1.

According to this first variant, when hydrogen peroxide is used as oxidizing agent, it is advantageously used in a hydrogen peroxide:hydrogen sulphide weight ratio varying from 1:1 to 3:1. Preferably, it is used in a hydrogen peroxide:hydrogen sulphide weight ratio of 1:1.

The oxidizing agent may be introduced in any form Preferably, it is introduced in the form of an aqueous solution.

According to this first variant, when sodium hypochlorite is used as oxidizing agent in the form of an aqueous solution, the sodium hypochlorite concentration of the latter is advantageously between 10 and 15% by weight. Preferably, it is of the order of 12.5% by weight.

According to this first variant, when hydrogen peroxide is used as oxidizing agent in the form of an aqueous solution, the hydrogen peroxide concentration of the latter is advantageously between 35 and 70% by weight. Preferably, it is of the order of 50% by weight.

According to this first variant, when hydrogen peroxide is used as oxidizing agent, the aqueous effluent resulted from the oxidation step is preferably subjected to a flocculation-decantation step in order to remove therefrom the insoluble and colloidal sulphur formed, before being discharged

According to a second variant of the process according to the invention, the oxidation step aimed at removing H₂S advantageously consists in the destruction of H₂S via the introduction of an oxidizing agent at the alkaline washing step, preferably in the washing column. Advantageously, the alkaline washing step takes place after the aqueous quenching step.

Any oxidizing agent may be used. There may be mentioned in particular hydrogen peroxide, sodium hypochlorite and the oxides of chlorine. Hydrogen peroxide and sodium hypochlorite are however preferred, with a most particular preference for hydrogen peroxide.

According to this second variant, when sodium hypochlorite is used as oxidizing agent, it is advantageously used in a sodium hypochlorite:sulphide ion molar ratio of 4:1.

According to this second variant, when hydrogen peroxide is used as oxidizing agent, it is advantageously used in a hydrogen peroxide:sulphide ion molar ratio of 4:1.

The oxidizing agent may be introduced in any form Preferably, it is introduced in the form of an aqueous solution.

According to this second variant, when sodium hypochlorite is used as oxidizing agent in the form of an aqueous solution, the sodium hypochlorite concentration of the latter is advantageously between 10 and 15% by weight. Preferably, it is of the order of 12.5% by weight.

According to this second variant, when hydrogen peroxide is used as oxidizing agent in the form of an aqueous solution, the hydrogen peroxide concentration of the latter is advantageously between 35 and 70% by weight. Preferably, it is of the order of 50% by weight.

The oxidizing agent may be introduced alone or as a mixture with NaOH. Preferably, it is introduced as a mixture with NaOH.

This variant has the advantage of making it possible to limit the number of operations and, in the case where hydrogen peroxide is the oxidizing agent, to avoid the formation of a sulphur colloid which risks coagulating and creating blockages since, in this case, it is the sulphates that are formed.

According to a third variant of the process according to the invention, the oxidation step aimed at removing H₂S advantageously consists in the destruction of H₂S via the introduction of an oxidizing agent into the alkaline solution derived from the alkaline washing step, preferably placed in an intermediate buffer reservoir. Advantageously, the alkaline washing step takes place after the aqueous quenching step.

Any oxidizing agent may be used. There may be mentioned in particular hydrogen peroxide, sodium hypochlorite and the oxides of chlorine. Hydrogen peroxide and sodium hypochlorite are however preferred, with a most particular preference for hydrogen peroxide.

According to this third variant, when sodium hypochlorite is used as oxidizing agent, it is advantageously used in a sodium hypochlorite:sulphide ion molar ratio of 4:1.

According to this third variant, when hydrogen peroxide is used as oxidizing agent, it is advantageously used in a hydrogen peroxide:sulphide ion molar ratio of 4:1.

The oxidizing agent may be introduced in any form Preferably, it is introduced in the form of an aqueous solution.

According to this third variant, when sodium hypochlorite is used as oxidizing agent in the form of an aqueous solution, the sodium hypochlorite concentration of the latter is advantageously between 10 and 15% by weight. Preferably, it is of the order of 12.5% by weight.

According to this third variant, when hydrogen peroxide is used as oxidizing agent in the form of an aqueous solution, the hydrogen peroxide concentration of the latter is advantageously between 35 and 70% by weight. Preferably, it is of the order of 50% by weight.

This variant has the advantage of allowing a limitation of the number of operations and, in the case where hydrogen peroxide is the oxidizing agent, to avoid the formation of a sulphur colloid which risks coagulating and creating blockages since, in this case, it is the sulphates that are formed.

This variant has the advantage of limiting the possibilities of undesirable effect of side reactions of the oxidizing agent in the medium of the cracking products essentially consisting of fuels or reactive products such as hydrogen, alkanes, alkenes and acetylene.

According to the three variants of the process according to the invention, the mixture of products subjected to the oxidation step is also advantageously subjected to the other treatment steps following the first cracking step. An alkaline solution consequently advantageously results therefrom in all cases.

The second and third variants of the process according to the invention are preferred with a most particular preference for the third variant.

Advantageously, the mixture of products containing ethylene and other constituents obtained in step b) comprises hydrogen, methane, compounds comprising from 2 to 7 carbon atoms, carbon monoxide, nitrogen and oxygen. The hydrogen, the methane and the compounds comprising from 2 to 7 carbon atoms other than acetylene are preferably present in an amount of at least 200 ppm by volume relative to the total volume of the said mixture of products. The carbon monoxide, the nitrogen, the oxygen and the acetylene may be present in an amount of less than 200 ppm by volume or in an amount of at least 200 ppm by volume relative to the total volume of the said mixture of products. Compounds containing more than 7 carbon atoms, carbon dioxide, hydrogen sulphide and water may also be present in the abovementioned mixture of products in an amount of less than 200 ppm by volume relative to the total volume of the said mixture of products.

After step b) defined above, the mixture of products containing ethylene and other constituents is subjected to step c) which advantageously comprises a maximum of four, preferably a maximum of three separation steps in order to obtain the fraction or fractions containing ethylene.

The separation of the mixture of products containing ethylene and other constituents in step c) leads to the formation of at least one fraction containing ethylene, preferably two fractions containing ethylene, in a particularly preferred manner one fraction containing ethylene which is enriched with the compounds lighter than ethylene, called below fraction A, and a second fraction containing ethylene, advantageously enriched with ethylene, called fraction B below, and a heavy fraction (fraction C).

According to the process according to the invention, fraction A is advantageously conveyed to the chlorination reactor and fraction B advantageously to the oxychlorination reactor, preferably after expansion with recovery of energy.

According to the process of the invention, the quantities defined below to characterize the fraction B and the fraction A are those before their respective entry into oxychlorination and into chlorination.

Fraction B is advantageously characterized by a hydrogen content of less than or equal to 2%, preferably of less than or equal to 0.5% and in a particularly preferred manner of less than or equal to 0.1% by volume relative to the total volume of fraction B.

Fraction B is characterized by a content of compounds containing at least 3 carbon atoms, advantageously less than or equal to 0.01%, preferably less than or equal to 0.005% and in a particularly preferred manner less than or equal to 0.001% by volume relative to the total volume of fraction B.

Fraction B advantageously contains from 40% to 99.5% by volume of ethylene relative to the total volume of fraction B. Fraction B advantageously contains at least 40%, preferably at least 50% and in a particularly preferred manner at least 60% by volume of ethylene relative to the total volume of fraction B. Fraction B advantageously contains at most 99.5%, preferably at most 99.2% and in a particularly preferred manner at most 99% by volume of ethylene relative to the total volume of fraction B.

In the preferred case where the hydrocarbon source is ethane, fraction B advantageously comprises at least 60%, preferably at least 70% and in a particularly preferred manner at least 75% by volume of ethylene relative to the total volume of fraction B. Fraction B advantageously comprises at most 99.5%, preferably at most 99.2% and in a particularly preferred manner at most 99% by volume of ethylene relative to the total volume of fraction B.

In the preferred case where the hydrocarbon source is a propane/butane mixture, fraction B advantageously comprises at least 40%, preferably at least 50% and in a particularly preferred manner at least 60% by volume of ethylene relative to the total volume of fraction B. Fraction B advantageously comprises at most 99.5%, preferably at most 99.2% and in a particularly preferred manner at most 99% by volume of ethylene relative to the total volume of fraction B.

Fraction B is additionally characterized by an acetylene content which is advantageously less than or equal to 0.01%, preferably less than or equal to 0.005% and in a particularly preferred manner less than or equal to 0.001% by volume relative to the total volume of fraction B.

Fraction A is advantageously enriched with compounds which are lighter than ethylene. These compounds are generally methane, nitrogen, oxygen, hydrogen and carbon monoxide. Advantageously, fraction A contains at least 70%, preferably at least 80% and in a particularly preferred manner at least 85% of compounds lighter than ethylene which are contained in the mixture of products subjected to step b). Advantageously, fraction A contains at most 99.99%, preferably at most 99.97% and in a particularly preferred manner at most 99.95% of compounds lighter than ethylene which are contained in the mixture of products subjected to step b).

In the preferred case where the hydrocarbon source is ethane, fraction A contains at least 90%, preferably at least 95% and in a particularly preferred manner at least 98% of compounds lighter than ethylene which are contained in the mixture of products subjected to step b). Advantageously, fraction A contains at most 99.99%, preferably at most 99.98% and in a particularly preferred manner at most 99.97% of compounds lighter than ethylene which are contained in the mixture of products subjected to step b).

In the preferred case where the hydrocarbon source is a propane/butane mixture, fraction A contains at least 70%, preferably at least 80% and in a particularly preferred manner at least 85% of compounds lighter than ethylene which are contained in the mixture of products subjected to step b). Advantageously, fraction A contains at most 99.99%, preferably at most 99.95% and in a particularly preferred manner at most 99.9% of compounds lighter than ethylene which are contained in the mixture of products subjected to step b).

Fraction A is characterized by a content of compounds containing at least 3 carbon atoms, advantageously less than or equal to 0.01%, preferably less than or equal to 0.005% and in a particularly preferred manner less than or equal to 0.001% by volume relative to the total volume of fraction A.

Fraction A advantageously contains a content by volume of ethylene such that it represents from 10% to 90% of the content by volume of ethylene of fraction B. Fraction A advantageously contains a content by volume of ethylene such that it is less than or equal to 90%, preferably less than or equal to 85% and in a particularly preferred manner less than or equal to 80% of the content by volume of ethylene of fraction B. Fraction A advantageously contains a content by volume of ethylene such that it is at least 10%, preferably at least 15% and in a particularly preferred manner at least 20% of the content by volume of ethylene of fraction B.

In the preferred case where the hydrocarbon source is ethane, fraction A advantageously contains a content by volume of ethylene such that it is less than or equal to 90%, preferably less than or equal to 85% and in a particularly preferred manner less than or equal to 80% of the content by volume of ethylene of fraction B. Fraction A advantageously contains a content by volume of ethylene such that it is at least 15%, preferably at least 20% and in a particularly preferred manner at least 22% of the content by volume of ethylene of fraction B.

In the preferred case where the hydrocarbon source is a propane/butane mixture, fraction A advantageously contains a content by volume of ethylene such that it is less than or equal to 80%, preferably less than or equal to 75% and in a particularly preferred manner less than or equal to 70% of the content by volume of ethylene of fraction B. Fraction A advantageously contains a content by volume of ethylene such that it is at least 10%, preferably at least 15% and in a particularly preferred manner at least 20% of the content by volume of ethylene of fraction B.

Fraction A is additionally characterized by an acetylene content which is advantageously less than or equal to 0.01%, preferably less than or equal to 0.005% and in a particularly preferred manner less than or equal to 0.001% by volume relative to the total volume of fraction A.

According to a first embodiment of the process according to the invention, considering that the process for the manufacture of DCE is advantageously balanced (that is to say that the process of manufacture by chlorination and oxychlorination of ethylene and pyrolysis of the 1,2-dichloroethane (DCE) formed makes it possible to generate the quantity of HCl necessary for the process), the fraction by weight of the ethylene throughput in each of fractions A and B is advantageously between 45 and 55% of the total quantity of ethylene produced (fraction A+fraction B). Preferably, the fraction by weight of the throughput of ethylene in fraction A is of the order of 55% and the fraction by weight of the throughput of ethylene in fraction B is of the order of 45% of the total quantity produced. In a particularly preferred manner, the fraction by weight of the throughput of ethylene in fraction A is of the order of 52.5% and the fraction by weight of the throughput of ethylene in fraction B is of the order of 47.5% of the total quantity produced.

According to a second embodiment of the process according to the invention, considering that the process for the manufacture of DCE is advantageously unbalanced (that is to say for example that an external source of HCl makes it possible to provide part of the supply of HCl for the oxychlorination or that a fraction of the DCE produced is not subjected to pyrolysis), the fraction by weight of the throughput of ethylene in each of fractions A and B is advantageously between 20 and 80% of the total quantity of ethylene produced (fraction A+fraction B). Preferably, the fraction by weight of the throughput of ethylene in fraction A is between 25 and 75% of the total quantity of ethylene produced (fraction A+fraction B).

According to a first variant of the second embodiment of the process according to the invention, considering that the process for the manufacture of DCE is advantageously unbalanced by an external source of HCl, the fraction by mole of the throughput of ethylene in fraction A is advantageously between 45 and 55%, preferably between 50 and 54% and in a particularly preferred manner of the order of 52.5% of the difference between the total molar quantity of ethylene contained in the mixture of products subjected to step b) and the molar quantity of HCl of the external source.

According to a second variant of the second embodiment of the process according to the invention, considering that the process for the manufacture of DCE is advantageously unbalanced by a co-production of DCE (some of the DCE is therefore not subjected to pyrolysis), the fraction by mole of the throughput of ethylene in fraction B is advantageously between 45 and 55%, preferably between 46 and 50% and in a particularly preferred manner of the order of 47.5% of the difference between the total molar quantity of ethylene contained in the mixture of products subjected to step b) and the molar quantity of DCE co-produced.

During step c), the mixture of products is preferably separated into at least one fraction containing ethylene and into a heavy fraction (fraction C). Fraction C advantageously contains ethane and compounds comprising at least 3 carbon atoms. Advantageously, these compounds comprising at least 3 carbon atoms result from the mixture of products containing ethylene and other constituents derived from step b) or are generated by side reactions during step c). Among the compounds comprising at least 3 carbon atoms, there may be mentioned propane, propene, butanes and their unsaturated derivatives as well as all the saturated or unsaturated heavier compounds.

Any separation process may be used to separate the said mixture of products containing ethylene into fraction A, fraction B and fraction C provided that it advantageously comprises a maximum of four, preferably a maximum of three separation steps in order to obtain both fractions A and B.

According to a first preferred mode of separation, the mixture of products containing ethylene derived from step b) is subjected to a first separation step which makes it possible to extract fraction C therefrom and the resulting mixture is then subjected to a second step for separation into fraction A and fraction B.

According to a second preferred mode of separation, the mixture of products containing ethylene derived from step b) is subjected to a first separation step which makes it possible to extract fraction A therefrom and the resulting mixture is then subjected to a second step for separation into fraction B and fraction C.

The first mode of separation is particularly preferred. Numerous variants can make it possible to carry out this first particularly preferred mode of separating the mixture of products containing ethylene derived from step a).

A preferred variant of the first mode of separation consists in subjecting the said mixture to a first separation step aimed at extracting fraction C and then in subjecting the resulting mixture to a second step for separation into fraction A and fraction B which are both distillation steps performed by means of a distillation column equipped with the associated auxiliary equipment such as at least one reboiler and at least one condenser.

According to this preferred variant of the first mode of separation, fraction C advantageously leaves at the bottom of the first distillation column, fraction A at the top of the second distillation column and fraction B at the bottom of the second distillation column.

The distillation column may be chosen from plate distillation columns, packed distillation columns, distillation columns with structured packing and distillation columns combining two or more of the abovementioned internals.

The chlorination reaction is advantageously performed in a liquid phase (preferably mainly DCE) containing a dissolved catalyst such as FeCl₃ or another Lewis acid. It is possible to advantageously combine this catalyst with cocatalysts such as alkali metal chlorides. A pair which has given good results is the complex of FeCl₃ with LiCl (lithium tetrachloroferrate—as described in patent application NL 6901398).

The quantities of FeCl₃ advantageously used are of the order of 1 to 10 g of FeCl₃ per kg of liquid stock. The molar ratio of FeCl₃ to LiCl is advantageously of the order of 0.5 to 2.

The chlorination process according to the invention is advantageously performed at temperatures of between 30 and 150° C. Good results were obtained regardless of the pressure both at a temperature less than the boiling temperature (under-cooled chlorination) and at the boiling temperature itself (boiling chlorination).

When the chlorination process according to the invention is a under-cooled chlorination, it gave good results by operating at a temperature which is advantageously greater than or equal to 50° C. and preferably greater than or equal to 60° C., but advantageously less than or equal to 80° C. and preferably less than or equal to 70° C.; with a pressure in the gaseous phase advantageously greater than or equal to 1.5 and preferably greater than or equal to 2 absolute bar, but advantageously less than or equal to 20, preferably less than or equal to 10 and in a particularly preferred manner less than or equal to 6 absolute bar.

A boiling chlorination process is particularly preferred which makes it possible, where appropriate, to usefully recover the heat of reaction. In this case, the reaction advantageously takes place at a temperature greater than or equal to 60° C., preferably greater than or equal to 90° C. and in a particularly preferred manner greater than or equal to 95° C. but advantageously less than or equal to 150° C. and preferably less than or equal to 135° C.; with a pressure in the gaseous phase advantageously greater than or equal to 0.2, preferably greater than or equal to 0.5, in a particularly preferred manner greater than or equal to 1.2 and in a most particularly preferred manner greater than or equal to 1.5 absolute bar but advantageously less than or equal to 10 and preferably less than or equal to 6 absolute bar.

The chlorination process may also be a loop under-cooled boiling mixed chlorination process. The expression loop under-cooled boiling mixed chlorination process is understood to mean a process in which cooling of the reaction medium is performed, for example, by means of an exchanger immersed in the reaction medium or by a loop circulating in an exchanger, while producing in a gaseous phase at least the quantity of DCE formed. Advantageously, the reaction temperature and pressure are adjusted for the DCE produced to leave in the gaseous phase and to remove the remainder of the calories from the reaction medium by means of the exchange surface.

In addition, the chlorination process is advantageously performed in a chlorinated organic liquid medium. Preferably, this chlorinated organic liquid medium, also called liquid stock, mainly consists of DCE.

The fraction A containing the ethylene and the chlorine (itself pure or diluted) may be introduced by any known device into the reaction medium together or separately. A separate introduction of the fraction A may be advantageous in order to increase its partial pressure and facilitate its dissolution which often constitutes a limiting step of the process.

The chlorine is added in a sufficient quantity to convert most of the ethylene and without requiring the addition of an excess of unconverted chlorine. The chlorine/ethylene ratio used is preferably between 1.2 and 0.8 and in a particularly preferred manner between 1.05 and 0.95 mol/mol.

The chlorinated products obtained contain mainly DCE and small quantities of by-products such as 1,1,2-trichloroethane or small quantities of chlorination products of ethane or methane. The separation of the DCE obtained from the stream of products derived from the chlorination reactor is carried out according to known modes and makes it possible in general to exploit the heat of the chlorination reaction.

The unconverted products (methane, carbon monoxide, nitrogen, oxygen and hydrogen) are then advantageously subjected to an easier separation than what would have been necessary to separate pure ethylene starting with the initial mixture.

The DCE leaving the chlorination containing chlorine is advantageously subjected to an alkaline washing. This alkaline washing step advantageously uses the alkaline solution resulting from the process according to the invention.

The oxychlorination reaction is advantageously performed in the presence of a catalyst comprising active elements including copper deposited on an inert support. The inert support is advantageously chosen from alumina, silica gels, mixed oxides, clays and other supports of natural origin. Alumina constitutes a preferred inert support.

Catalysts comprising active elements which are advantageously at least two in number, one of which is copper, are preferred. Among the active elements other than copper, there may be mentioned alkali metals, alkaline-earth metals, rare-earth metals and metals of the group consisting of ruthenium, rhodium, palladium, osmium, iridium, platinum and gold. The catalysts containing the following active elements are particularly advantageous: copper/magnesium/potassium, copper/magnesium/sodium; copper/magnesium/lithium, copper/magnesium/caesium, copper/magnesium/sodium/lithium, copper/magnesium/potassium/lithium and copper/magnesium/caesium/lithium, copper/magnesium/sodium/potassium, copper/magnesium/sodium/caesium and copper/magnesium/potassium/caesium. The catalysts described in patent applications EP-A 255 156, EP-A 494 474, EP-A-657 212 and EP-A 657 213, incorporated by reference, are most particularly preferred.

The copper content, calculated in metal form, is advantageously between 30 and 90 g/kg, preferably between 40 and 80 g/kg and in a particularly preferred manner between 50 and 70 g/kg of catalyst.

The magnesium content, calculated in metal form, is advantageously between 10 and 30 g/kg, preferably between 12 and 25 g/kg and in a particularly preferred manner between 15 and 20 g/kg of catalyst.

The alkali metal content, calculated in metal form, is advantageously between 0.1 and 30 g/kg, preferably between 0.5 and 20 g/kg and in a particularly preferred manner between 1 and 15 g/kg of catalyst.

The Cu:Mg:alkali metal(s) atomic ratios are advantageously 1:0.1-2:0.05-2, preferably 1:0.2-1.5:0.1-1.5 and in a particularly preferred manner 1:0.5-1:0.15-1.

Catalysts having a specific surface area, measured according to the B.E.T. method with nitrogen, advantageously between 25 m²/g and 300 m²/g, preferably between 50 and 200 m²/g and in a particularly preferred manner between 75 and 175 m²/g, are particularly advantageous.

The catalyst may be used in a fixed bed or in a fluidized bed. This second option is preferred. The oxychlorination process is exploited under the range of the conditions usually recommended for this reaction. The temperature is advantageously between 150 and 300° C., preferably between 200 and 275° C. and most preferably from 215 to 255° C. The pressure is advantageously greater than atmospheric pressure. Values of between 2 and 10 absolute bar gave good results. The range between 4 and 7 absolute bar is preferred. This pressure may be usefully modulated in order to obtain an optimum residence time in the reactor and to maintain a constant rate of passage for various speeds of operation. The usual residence times range from 1 to 60 seconds and preferably from 10 to 40 seconds.

The source of oxygen for this oxychlorination may be air, pure oxygen or a mixture thereof, preferably pure oxygen. The latter solution, which allows easy recycling of the unconverted reagents, is preferred.

The reagents may be introduced into the bed by any known device. It is generally advantageous to introduce the oxygen separately from the other reagents for safety reasons. These also require maintaining the gaseous mixture leaving the reactor or recycled thereto outside the limits of inflammability at the pressures and temperatures considered. It is preferable to maintain a so-called rich mixture, that is containing too little oxygen relative to the fuel to ignite. In this regard, the abundant presence (>2%, preferably>5% vol) of hydrogen would constitute a disadvantage given the wide range of inflammability of this compound.

The hydrogen chloride/oxygen ratio used is advantageously between 2 and 4 mol/mol. The ethylene/hydrogen chloride ratio is advantageously between 0.4 and 0.6 mol/mol.

The chlorinated products obtained contain mainly DCE and small quantities of by-products such as 1,1,2-trichloroethane. The separation of the DCE obtained from the stream of products derived from the oxychlorination reactor is carried out according to known modes. The heat of the oxychlorination reaction is generally recovered in vapour form which can be used for the separations or for any other purpose.

The unconverted products such as methane and ethane are then subjected to an easier separation than that which would have been necessary to separate pure ethylene starting from the initial mixture.

The crude gases from the oxychlorination advantageously undergo an alkaline washing aimed at destroying the unconverted HCl. This alkaline washing step, advantageously using the alkaline solution resulting from the process according to the invention, may be carried out in one or two steps. A device is preferred in which the first washing step occurs in an acidic medium, with a second washer supplied with slightly alkaline solution in order to destroy the last traces of HCl. In this application, it is not desired to completely destroy the CO₂ which is not problematic. The conveying of partially exhausted alkali from the second step to the first is particularly preferred in order to fully exploit the capacity for fixing HCl.

The DCE obtained is then separated from the streams of products derived from the chlorination and oxychlorination reactors and conveyed to the pyrolysis oven so as to be advantageously converted to VC therein.

The invention therefore also relates to a process for the manufacture of VC. To this effect, the invention relates to a process for the manufacture of VC, characterized in that the DCE obtained by the process according to the invention is subjected to pyrolysis.

The conditions under which the pyrolysis may be carried out are known to persons skilled in the art. This pyrolysis is advantageously obtained by a reaction in the gaseous phase in a tubular oven. The usual pyrolysis temperatures are between 400 and 600° C. with a preference for the range between 480° C. and 540° C. The residence time is advantageously between 1 and 60 s with a preference for the range from 5 to 25 s. The rate of conversion of the DCE is advantageously limited to 45 to 75% in order to limit the formation of by-products and the fouling of the tubes of the oven. The following steps make it possible, using any known device, to collect the purified VC and the hydrogen chloride to be upgraded preferably to the oxychlorination. Following purification, the unconverted DCE is advantageously conveyed to the pyrolysis oven.

In addition, the invention also relates to a process for the manufacture of PVC. To this effect, the invention relates to a process for the manufacture of PVC by polymerization of the VC obtained by the process according to the invention.

The process for the manufacture of PVC may be a mass, solution or aqueous dispersion polymerization process, preferably it is an aqueous dispersion polymerization process.

The expression aqueous dispersion polymerization is understood to mean free radical polymerization in aqueous suspension as well as free radical polymerization in aqueous emulsion and polymerization in aqueous microsuspension.

The expression free radical polymerization in aqueous suspension is understood to mean any free radical polymerization process performed in aqueous medium in the presence of dispersing agents and oil-soluble free radical initiators.

The expression free radical polymerization in aqueous emulsion is understood to mean any free radical polymerization process performed in aqueous medium in the presence of emulsifying agents and water-soluble free radical initiators.

The expression aqueous microsuspension polymerization, also called polymerization in homogenized aqueous dispersion, is understood to mean any free radical polymerization process in which oil-soluble initiators are used and an emulsion of droplets of monomers is prepared by virtue of a powerful mechanical stirring and the presence of emulsifying agents.

The alkaline solution generated during the alkaline washing step of the process for the manufacture of DCE according to the invention may be advantageously used to neutralize any acidic effluent from the installation for producing DCE, VC and PVC.

Thus, the subject of the invention is also the use of the alkaline solution obtained during the alkaline washing step of the process for the manufacture of DCE according to the invention in order to neutralize any acidic effluent from the processes for the manufacture of DCE, VC and PVC according to the invention.

As acidic effluents which may be treated by means of the said alkaline solutions, there may be mentioned the crude gases leaving the chlorination or the oxychlorination and mainly containing DCE, HCl, for example not converted during oxychlorination and preferably anhydrous, chlorine, but also the incineration residues.

One advantage of the process is that it solves the problem of removing the sulphides normally present in the effluent from cracking.

Another advantage of the process according to the invention is that it makes it possible to have an alkaline effluent composed of carbonate and sulphate which may be used with no disadvantage in the manufacture of DCE and VCM.

Finally, one last advantage of the process according to the invention is that it makes it possible to have, on the same industrial site, a completely integrated process from the hydrocarbon source to the polymer obtained starting with the monomer manufactured.

1-18. (canceled)

19. A process for the manufacture of 1,2-dichloroethane starting with a hydrocarbon source according to which:
a) the hydrocarbon source is subjected to a first cracking step, namely a pyrolysis step carried out in a cracking oven, thus producing a mixture of cracking products;
b) the said mixture of cracking products is subjected to a succession of treatment steps which make it possible to obtain a mixture of products containing ethylene and other constituents, among which an aqueous quenching step, an alkaline washing step aimed at removing at least most of the carbon dioxide generating an alkaline solution and an oxidation step aimed at removing the hydrogen sulphide contained in the mixture of cracking products;
c) the mixture of products containing ethylene derived from step b) is separated into at least one fraction containing ethylene and into a heavy fraction;
d) the fraction(s) containing the ethylene is (are) conveyed to a chlorination reactor and/or an oxychlorination reactor, in which reactors most of the ethylene present is converted to 1,2-dichloroethane;
e) the 1,2-dichloroethane obtained is separated from the streams of products derived from the chlorination and oxychlorination reactors.

20. The process according to claim 19, wherein the hydrocarbon source is chosen from the group consisting of naphtha, gas oil, natural gas liquid, ethane, propane, butane, isobutane and mixtures thereof.

21. The process for the manufacture of 1,2-dichloroethane according to claim 19, wherein the hydrocarbon source is chosen from the group consisting of ethane, propane, butane and propane/butane mixtures.

22. The process according to claim 19, wherein the oxidation step aimed at removing hydrogen sulphide consists in destroying the hydrogen sulphide via the introduction of an oxidizing agent in the aqueous quenching step.

23. The process according to claim 19, wherein the oxidation step aimed at removing hydrogen sulphide consists in destroying hydrogen sulphide via the introduction of an oxidizing agent in the alkaline washing step.

24. The process according to claim 19, wherein the oxidation step aimed at removing hydrogen sulphide consists in destroying hydrogen sulphide via the introduction of an oxidizing agent into the alkaline solution derived from the alkaline washing step.

25. The process according to claim 22, wherein the oxidizing agent is hydrogen peroxide.

26. The process according to claim 23, wherein the oxidizing agent is hydrogen peroxide.

27. The process according to claim 24, wherein the oxidizing agent is hydrogen peroxide.

28. The process according to claim 19, wherein the mixture of products containing ethylene and other constituents derived from step b) comprises hydrogen, methane, compounds comprising from 2 to 7 carbon atoms, carbon monoxide, nitrogen and oxygen.

29. The process according to claim 19, wherein the separation of the mixture of products containing ethylene and other constituents in step c) leads to the formation of a fraction enriched with the compounds lighter than ethylene containing part of the ethylene (fraction A), a fraction enriched with ethylene (fraction B) and a heavy fraction (fraction C).

30. The process according to claim 29, wherein fraction B contains from 40% to 99.5% by volume of ethylene relative to the total volume of fraction B.

31. The process according to claim 29, wherein fraction A contains a content by volume of ethylene such that it represents from 10% to 90% of the content by volume of ethylene of fraction B.

32. A process for the manufacture of vinyl chloride, wherein the 1,2-dichloroethane obtained by the process according to claim 19 is subjected to pyrolysis.

33. A process for the manufacture of polyvinyl chloride by polymerization of the vinyl chloride obtained by the process according to claim 32.

34. Use of the alkaline solution obtained during the alkaline washing step of the process for the manufacture of 1,2-dichloroethane according to claim 19 for neutralizing any acidic effluent from the processes for the manufacture of 1,2-dichloroethane according to claim 19.

35. Use of the alkaline solution obtained during the alkaline washing step of the process for the manufacture of 1,2-dichloroethane according to claim 19 for neutralizing any acidic effluent from the processes for the manufacture of vinyl chloride.

36. Use of the alkaline solution obtained during the alkaline washing step of the process for the manufacture of 1,2-dichloroethane according to claim 19 for neutralizing any acidic effluent from the processes for the manufacture of polyvinyl chloride.
